(19) United States
(12) Patent Application Publication
(10) Pub. No.: US 2023/0414610 A1
(43) Pub. Date: Dec. 28, 2023

(54) THERAPIES FOR CANCER USING SMALL MOLECULES THAT BIND TO AND INHIBIT RAIL INTERACTING PROTEINS

(71) Applicant: Texas Tech University System, Lubbock, TX (US)

(72) Inventors: Sanjay Awasthi, Lubbock,, TX (US); Sharda Singh, Lubbock, TX (US)

(73) Assignee: Texas Tech University System, Lubbock, TX (US)

(21) Appl. No.: 18/037,732

(22) PCT Filed: Oct. 29, 2021
(86) PCT No.: PCT/US2021/057218

§ 371 Date: May 18, 2023

Related U.S. Application Data
(60) Provisional application No. 63/115,190, filed on Nov. 18, 2020.

(51) Int. Cl. A61K 31/4985; A61K 31/502; A61K 31/4709; A61K 31/4439; A61K 31/444; A61K 31/4045; A61P 35/00

(52) U.S. Cl. CPC A61K 31/4985; A61K 31/502; A61K 31/4709; A61P 35/00; A61K 31/444; A61K 31/4045; A61K 31/4439

(57) ABSTRACT

Embodiments of the present disclosure pertain to methods of treating or preventing cancer in a subject by administering to the subject a composition that includes at least one of the compounds disclosed herein. The cancer to be treated or prevented can include, without limitation, lung cancer, breast cancer, cancers associated with an over-expression of a ral interacting protein, or combinations thereof. Further embodiments of the present disclosure pertain to the compositions of the present disclosure.

### CROSS-REFERENCE TO RELATED APPLICATIONS

This application claims priority to U.S. Provisional Patent Application No. 63/115,190, filed on Nov. 18, 2020. The entirety of the aforementioned application is incorporated herein by reference.

### STATEMENT REGARDING FEDERALLY SPONSORED RESEARCH

This invention was made with government support under BC1711159-BC awarded by the Department of Defense. The government has certain rights in the invention.

### BACKGROUND

Current therapies for various types of cancers are generally unsatisfactory due to high rates of failure and excessive associated side effects. Numerous embodiments of the present disclosure address the aforementioned limitations.

### SUMMARY

In an embodiment, the present disclosure relates to a method of treating or preventing cancer in a subject. In some embodiments, the method includes administering to the subject a composition. In some embodiments, the composition can include at least one compound. In some embodiments, the at least one compound includes, without limitation:

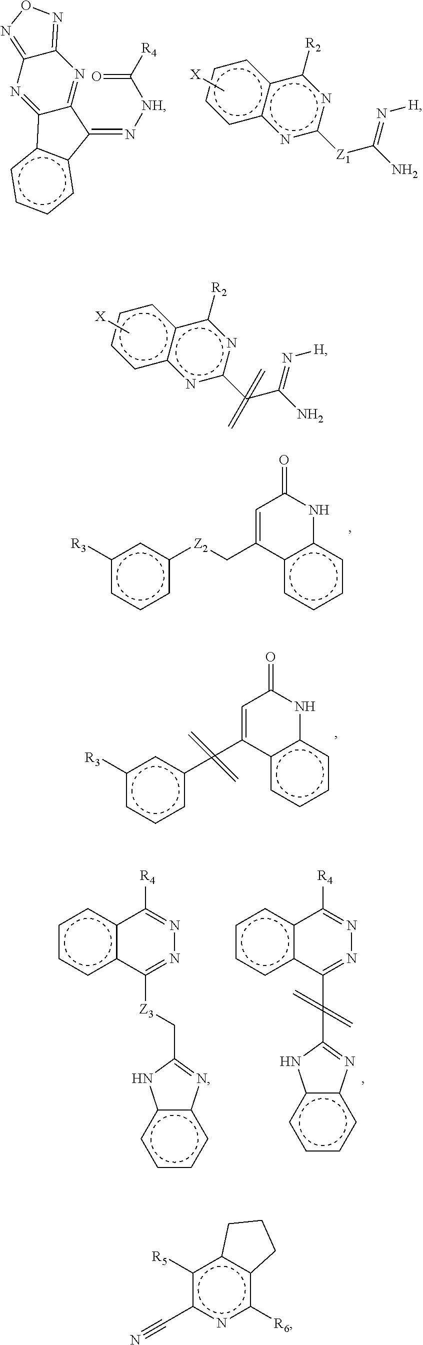

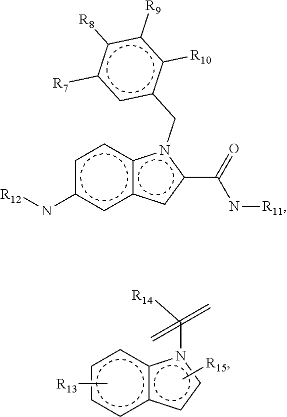

derivatives thereof, or combinations thereof.

In some embodiments, the cancer to be treated includes, without limitation, lung cancer, breast cancer, cancers associated with an over-expression of a ral interacting protein, or combinations thereof. Additional embodiments of the present disclosure pertain to the compositions of the present disclosure.

### DESCRIPTION OF THE DRAWINGS

FIG. 1 illustrates a method of treating or preventing a cancer in a subject by administering the compositions of the present disclosure to the subject.

FIG. 2 illustrates an embodiment of a compound of the present disclosure termed TTUHSC-SS-A4 and corresponding data related to breast cancer cell inhibition.

FIG. 3 illustrates an embodiment of a compound of the present disclosure termed TTUHSC-SS-B9 and corresponding data related to breast cancer cell inhibition.

FIG. 4 illustrates an embodiment of a compound of the present disclosure termed TTUHSC-SS-C2 and corresponding data related to breast cancer cell inhibition.

FIG. 5 illustrates an embodiment of a compound of the present disclosure termed TTUHSC-SS-C4 and corresponding data related to breast cancer cell inhibition.

FIG. 6 illustrates an embodiment of a compound of the present disclosure termed TTUHSC-SS-E4 and corresponding data related to breast cancer cell inhibition.

FIG. 7A illustrates images of nude mice after they were injected with MDA-MB-231 Luc2 cells and treated with 1 nmole single i.v. dose Mcule compounds A4, B7, and B9.

FIG. 7B illustrates results from Mcule compound testing.

FIG. 8 illustrates an embodiment of a compound of the present disclosure termed TTUHSC-SS-A4 and corresponding data related to lung cancer cell inhibition.

FIG. 9 illustrates an embodiment of a compound of the present disclosure termed TTUHSC-SS-B9 and corresponding data related to lung cancer cell inhibition.

FIG. 10 illustrates an embodiment of a compound of the present disclosure termed TTUHSC-SS-C2 and corresponding data related to lung cancer cell inhibition.

FIG. 11 illustrates an embodiment of a compound of the present disclosure termed TTUHSC-SS-C4 and corresponding data related to lung cancer cell inhibition.

FIG. 12 illustrates an embodiment of a compound of the present disclosure termed TTUHSC-SS-E4 and corresponding data related to lung cancer cell inhibition.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current therapies for cancers are generally unsatisfactory because of a high rate of failure and excessive side effects. Side effects are generally significant and result, in part, because few current therapies are targeted to cancerous cells alone.

For instance, breast cancer is the second leading cause of death in women after lung cancer and is the most common cancer among women worldwide. Hormone receptors inside and on the surface of healthy breast cells receive messages from the hormones estrogen and progesterone. The hormones attach to the receptors and help the cells continue to grow and function well. Some, but not all, breast cancers have at least one of these hormone receptors, while a smaller percentage of breast cancers produce an excessive amount of HER2 protein, thereby causing cells to grow and divide too quickly.

However, about 10-20% of breast cancers, called triple-negative breast cancer (TNBC), test negative for estrogen and progesterone receptors and excess HER2 protein. The cancerous growth that results is not fueled by the estrogen or progesterone hormones, or by the HER2 protein. Therefore, triple-negative breast cancer does not respond to medications targeting these hormones and protein receptors.

Triple-negative breast cancer is considered to be more aggressive and have a poorer prognosis than other types of breast cancer as fewer targeted medicines for treatment exist. Triple-negative breast cancer also tends to be a higher grade than other types of breast cancer, meaning that its cancer cells do not closely resemble normal, healthy breast cells in appearance and growth patterns. Similarly, triple-negative breast cancer cells are described as being “basal-like”, meaning that the cells more closely resemble the basal cells of the breast ducts and tend to be more aggressive.

Current methods of treatment for triple-negative breast cancer generally involve a combination of surgery, radiation therapy, chemotherapy, endocrine (hormone) therapy, and targeted therapy. Because of triple-negative breast cancer's lack of targeted use of therapies and aggressive nature, surgical decisions rely on more traditional clinicopathological variables, such as, but not limited to, patient age, tumor size, tumor grade, and patient preference. Similarly, because triple-negative breast cancers are aggressive and rapidly growing cancers, performing breast-conserving surgery followed by radiation therapy in the early stage of this disease may equate to a mastectomy.

Studies suggest that neoadjuvant chemotherapy, or treating triple-negative breast cancer with chemotherapy before surgery, improves patient prognosis and survival rates. Another method of treatment involves utilizing poly adenosine diphosphate (ADP)-ribose polymerase (PARP) enzyme inhibitors, which block DNA repair in both healthy and cancerous cells, and thereby makes some cancer cells less likely to survive their DNA damage. Similarly, utilizing immune checkpoint inhibitor medications help the immune system by targeting specific proteins that assist cancer cells in hiding from the immune system.

Similar therapies exist for other types of cancers. For instance, the main types of treatment for lung cancer are surgery (e.g., Pulmonary lobectomy, Wedge resection, Radiosurgery, and Video-assisted thoracoscopic), radiation therapy (RT), chemotherapy (CT), and immunotherapy.

However, current therapies for various types of cancers are generally unsatisfactory due to high rates of failure and excessive associated side effects. As a result, a need exists for developing therapies to treat breast cancer using targeted therapies that eliminate only cancerous cells and not healthy cells. Various embodiments of the present disclosure address the aforementioned need.

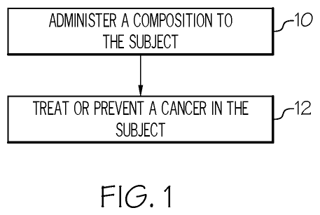

In some embodiments, the present disclosure relates to a method of treating or preventing cancer in a subject. In some embodiments illustrated in FIG. 1, the method includes administering to the subject a composition that includes one or more compounds (step 10). In some embodiments, the one or more compounds treats or prevents a cancer in a subject (step 12).

As set forth in more detail herein, the methods and compositions of the present disclosure can have numerous embodiments. For instance, the compositions of the present disclosure can include various compounds. Furthermore, various methods may be utilized to administer the compositions of the present disclosure to various subjects in order to treat and/or prevent cancer in such subjects through various mechanisms.

Anticancer Compositions

As set forth in more detail herein, the compositions of the present disclosure can include various compounds. For instance, in some embodiments, various functional groups, elements, and moieties can be included to form numerous compounds with numerous chemical configurations. In addition, the compositions of the present disclosure can have various advantageous properties.

In some embodiments, the compositions of the present disclosure can include at least one compound. In some embodiments, the at least one compound includes, without limitation:

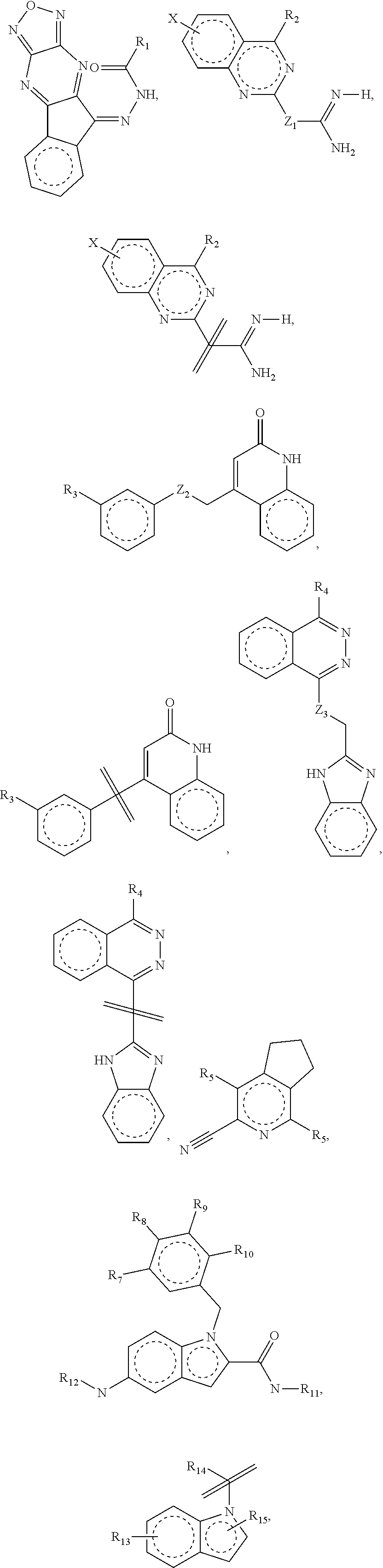

derivatives thereof, or combinations thereof.

In some embodiments, R₁, R₂, R₃, R₄, R₅, R₆, R₇, R₈, R₉, R₁₀, R₁₁, R₁₂, R₁₃, R₁₄, and R₁₅ are each independently selected from the group consisting of an alkane, an alkene, an alkyne, a benzene derivative, an alkyl, an alkenyl, an alkynyl, a phenol, a phenyl, a haloalkane, a fluoroalkane, a chloroalkane, a bromoalkane, an iodoalkane, an alcohol, a ketone, an aldehyde, an acyl halide, a carbonate, a carboxylic acid, an ester, a methoxy, a hydroperoxide, a peroxide, an ether, a hemiacetal, a hemiketal, an acetal, a ketal, an orthoester, a heterocycle, an orthocarbonate ester, an organic acid anhydride, a hydroxyl, a carbonyl, an aldehyde, a haloformyl, a carbonate ester, a carboxylate, a carboxyl, a carboalkoxy, a hydroperoxy, a peroxy, a methylenedioxy, a carboxylic anhydride, an amide, an amine, an imine, an imide, an azide, an azo, a cyanate, a nitrate, a nitrile, a nitrite, a nitro, a nitroso, an oxime, a pyridine, a pyridine derivative, a carbamate ester, a carbamate, a thiol, a sulfide, a disulfide, a sulfoxide, a sulfone, a sulfinic acid, a sulfonic acid, a sulfonate ester, a thiocyanate, a thioketone, a thial, a thiocarboxylic acid, a thioester, a dithiocarboxylic acid, a dithiocarboxylic acid ester, a phosphine, a phosphonic acid, a phosphate, a phosphodiester, H, C, F, CH₃, AcHN, CONH₂, CF₃, MeHN,

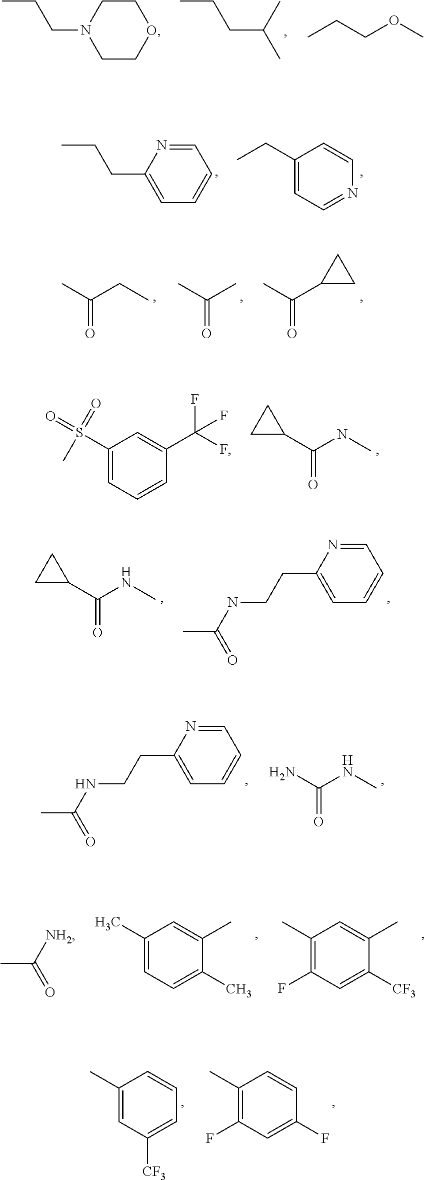

derivatives thereof, or combinations thereof.

In some embodiments, R₁, R₂, R₃, R₄, R₅, R₆, R₇, R₈, R₉, R₁₀, R₁₁, R₁₂, R₁₃, R₁₄, and R₁₅ can each independently include, without limitation, a phenol, a phenyl, a tetrazole, a pyridine, derivatives thereof, or combinations thereof. In some embodiments, R₁, R₂, R₃, R₄, R₅, R₆, R₇, R₈, R₉, R₁₀, R₁₁, R₁₂, R₁₃, R₁₄, and R₁₅ can each independently include, without limitation, H, C, F, CH₃, AcHN, CONH₂, CF₃, MeHN, derivatives thereof, or combinations thereof. In some embodiments, R₁, R₂, R₃, R₄, R₅, R₆, R₇, R₈, R₉, R₁₀, R₁₁, R₁₂, R₁₃, R₁₄, and R₁₅ can each independently include, without limitation:

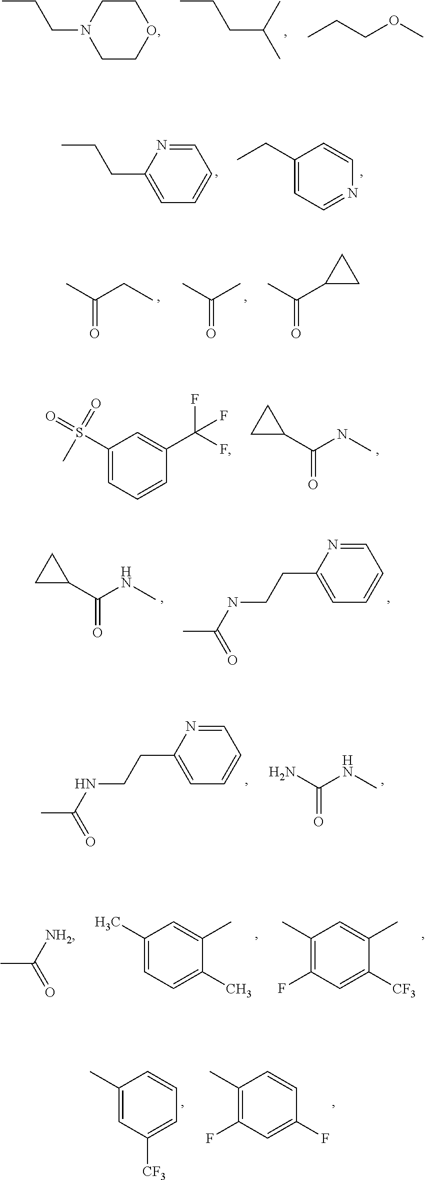

derivatives thereof, or combinations thereof.

In some embodiments, R₁, R₂, R₃, R₄, R₅, R₆, R₇, R₈, R₉, R₁₀, R₁₁, R₁₂, R₁₃, R₁₄, and R₁₅ can each independently include, without limitation, an alkane, an alkene, an alkyne, a benzene derivative, an alkyl, an alkenyl, an alkynyl, a phenyl, a haloalkane, a fluoroalkane, a chloroalkane, a bromoalkane, an iodoalkane, an alcohol, a ketone, an aldehyde, an acyl halide, a carbonate, a carboxylic acid, an ester, a methoxy, a hydroperoxide, a peroxide, an ether, a hemiacetal, a hemiketal, an acetal, a ketal, an orthoester, a heterocycle, an orthocarbonate ester, an organic acid anhydride, a hydroxyl, a carbonyl, an aldehyde, a haloformyl, a carbonate ester, a carboxylate, a carboxyl, a carboalkoxy, a methoxy, a hydroperoxy, a peroxy, an ether, a hemiacetal, a hemiketal, an acetal, a ketal, an orthoester, a methylenedioxy, an orthocarbonate ester, a carboxylic anhydride, an amide, an amine, an imine, an imide, an azide, an azo, a cyanate, a nitrate, a nitrile, a nitrite, a nitro, a nitroso, an oxime, a pyridine, a pyridine derivative, a carbamate ester, a carbamate, a thiol, a sulfide, a disulfide, a sulfoxide, a sulfone, a sulfinic acid, a sulfonic acid, a sulfonate ester, a thiocyanate, a thioketone, a thial, a thiocarboxylic acid, a thioester, a dithiocarboxylic acid, a dithiocarboxylic acid ester, a phosphine, a phosphonic acid, a phosphate, a phosphodiester, derivatives thereof, or combinations thereof.

In some embodiments, Z₁, Z₂, and Z₃ can each independently include, without limitation, O, NH, CH₂, S, Se, or combinations thereof. In some embodiments, X can include, without limitation, H, a halogen, fluorine, chlorine, bromine, iodine, or combinations thereof.

In particular embodiments, the compositions of the present disclosure can include, without limitation one or more of the following compounds:

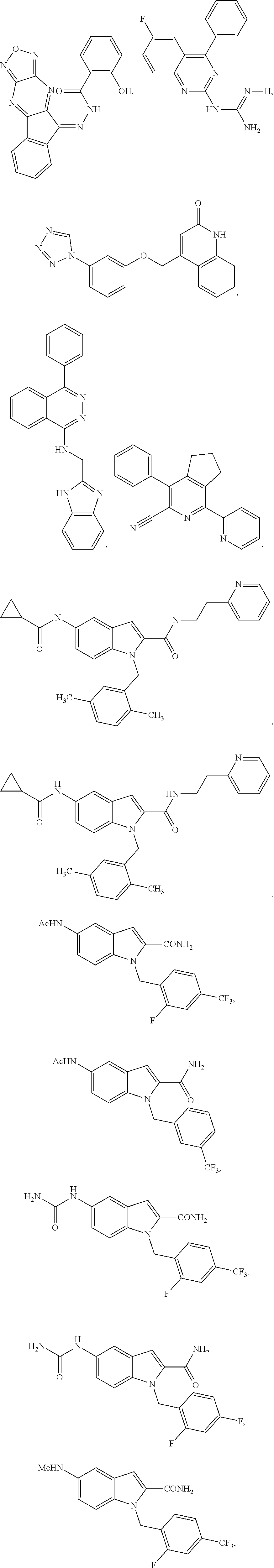

derivatives thereof, or combinations thereof.

In some embodiments, the composition can include, without limitation, the following compound:

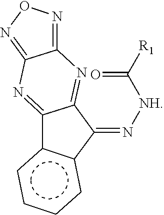

In some embodiments, R₁ is a phenol. In a particular embodiment, the composition can include, without limitation, the following compound:

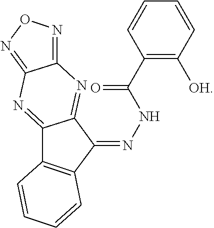 [SMILES: O=C(N/N=C1/c2ccccc2-c2nc3nonc3nc21)c1ccccc1O]

In some embodiments, the composition can include, without limitation, one or more of the following compounds:

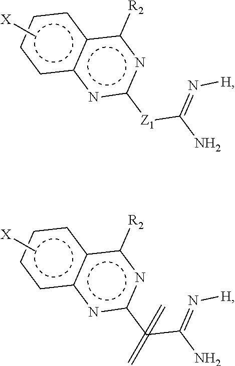

derivatives thereof, or combinations thereof.

In some embodiments, R₂ is a phenyl, X is fluorine, and Z₁ is NH. In a particular embodiment, the composition can include, without limitation, the following compound:

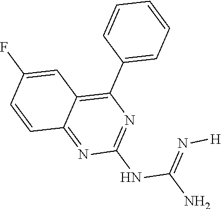 [SMILES: [H]/N=C(\N)Nc1nc(-c2ccccc2)c2cc(F)ccc2n1]

In some embodiments, the composition can include, without limitation, one or more of the following compounds:

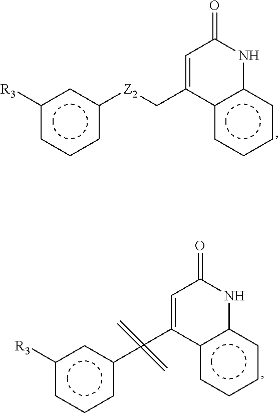

derivatives thereof, or combinations thereof.

In some embodiments, R₃ is a tetrazole and Z₂ is O. In a particular embodiment, the composition can include, without limitation, the following compound:

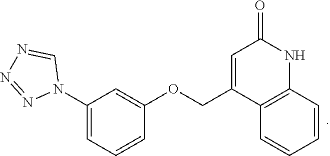 [SMILES: O=c1cc(COc2cccc(-n3cnnn3)c2)c2ccccc2[nH]1]

In some embodiments, the composition can include, without limitation, one or more of the following compounds:

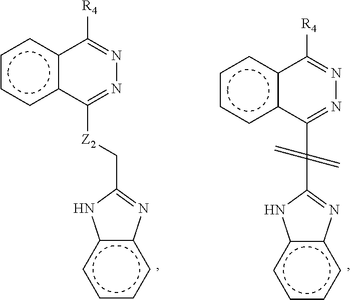

derivatives thereof, or combinations thereof.

In some embodiments, R₄ is a phenyl and Z₃ is NH. In a particular embodiment, the composition of the present disclosure can include, without limitation, the following compound:

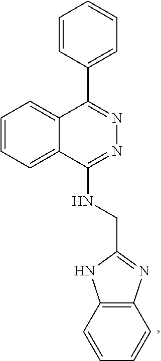 [SMILES: c1ccc(-c2nnc(NCc3nc4ccccc4[nH]3)c3ccccc23)cc1]

In some embodiments, the composition of the present disclosure can include, without limitation, the following compound:

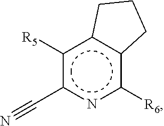

In some embodiments, R₅ is a phenyl and R₆ is a pyridine. In a particular embodiment, the composition can include, without limitation, the following compound:

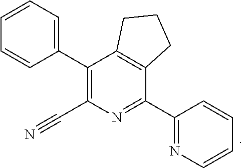 [SMILES: N#Cc1nc(-c2ccccn2)c2c(c1-c1ccccc1)CCC2]

In some embodiments, the composition of the present disclosure can include, without limitation, one or more of the following compounds:

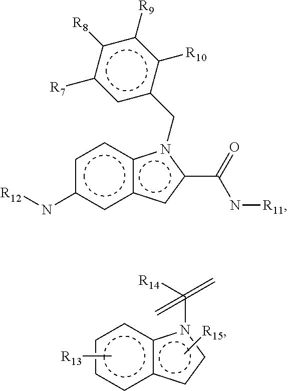

derivatives thereof, or combinations thereof.

In some embodiments, R₁₃ is

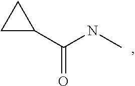

### R₁₄ is

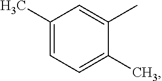

### and R₁₅ is

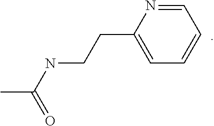

In a particular embodiment, the composition includes the following compound:

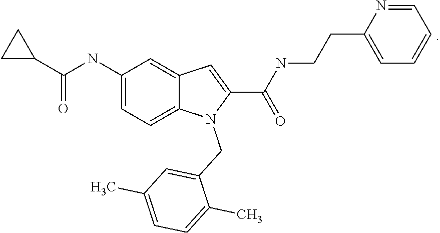 [SMILES: Cc1ccc(C)c(Cn2c(C(=O)NCCc3ccccn3)cc3cc(NC(=O)C4CC4)ccc32)c1]

In some embodiments, R₁₃ is

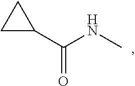

### R₁₄ is

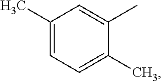

### and R₁₅ is

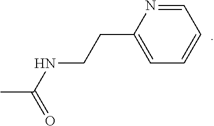

In a particular embodiment, the composition includes the following compound:

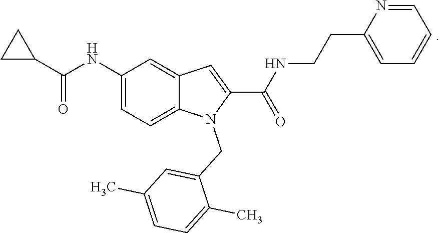 [SMILES: Cc1ccc(C)c(Cn2c(C(=O)NCCc3ccccn3)cc3cc(NC(=O)C4CC4)ccc32)c1]

In some embodiments, R₁₃ is AcHN, R₁₄ is

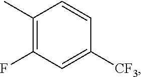

and R₁₅ is CONH₂. In a particular embodiment, the composition includes the following compound:

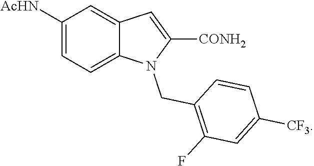 [SMILES: CC(=O)Nc1ccc2c(c1)cc(C(N)=O)n2Cc1ccc(C(F)(F)F)cc1F]

In some embodiments, R₁₃ is AcHN, R₁₄ is

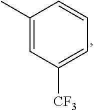

### and R₁₅ is

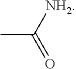

In a particular embodiment, the composition includes the following compound:

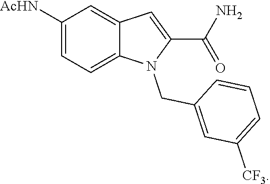 [SMILES: CC(=O)Nc1ccc2c(c1)cc(C(N)=O)n2Cc1cccc(C(F)(F)F)c1]

In some embodiments, R₁₃ is

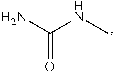

### R₁₄ is

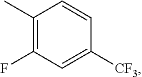

and R₁₅ is CONH₂. In a particular embodiment, the composition includes the following compound:

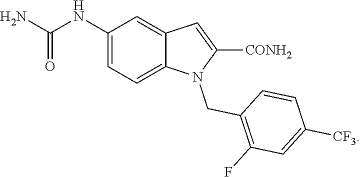 [SMILES: NC(=O)Nc1ccc2c(c1)cc(C(N)=O)n2Cc1ccc(C(F)(F)F)cc1F]

In some embodiments, R₁₃ is

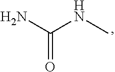

### R₁₄ is

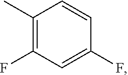

### and R₁₅ is

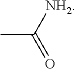

In a particular embodiment, the composition includes the following compound:

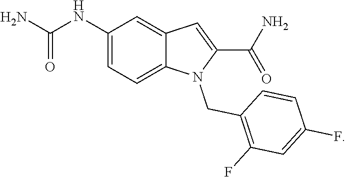 [SMILES: NC(=O)Nc1ccc2c(c1)cc(C(N)=O)n2Cc1ccc(F)cc1F]

In some embodiments, R₁₃ is MeHN, R₁₄ is

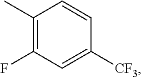

and R₁₅ is CONH₂. In a particular embodiment, the composition includes the following compound:

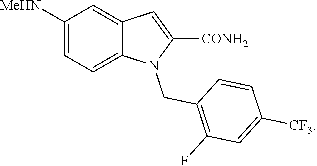 [SMILES: CNc1ccc2c(c1)cc(C(N)=O)n2Cc1ccc(C(F)(F)F)cc1F]

In some embodiments, the compositions of the present disclosure include one or more derivatives of any of the aforementioned compounds. In some embodiments, the one or more derivatives include one or more moieties that are derivatized with one or more functional groups. In some embodiments, the one or more moieties include carbon groups, nitrogen groups, or oxygen groups of the one or more compounds.

The moieties of the compounds of the present disclosure may be derivatized with various functional groups. For instance, in some embodiments, the functional groups include, without limitation, alkanes, alkenes, ethers, alkynes, alkoxyls, aldehydes, carboxyls, hydroxyls, hydrogens, sulfurs, phenyls, cyclic rings, aromatic rings, heterocyclic rings, linkers, or combinations thereof.

In some embodiments, the compositions of the present disclosure are associated with a delivery agent. In some embodiments, the delivery agent is a nanoparticle.

In some embodiments, the composition can include at least one excipient agent. In some embodiments, the at least one excipient agent can include, without limitation, anti-adherents, binders, coatings, colors, disintegrants, flavors, glidants, lubricants, preservatives, sorbents, sweeteners, vehicles, or combinations thereof.

In some embodiments, the compositions of the present disclosure can target various cell lines. In some embodiments, the compositions of the present disclosure bind to various biomolecules or macromolecules. In some embodiments, the biomolecules or macromolecules can include, without limitation, proteins.

In some embodiments, the compositions of the present disclosure target cancer cell lines by inhibiting or binding a ral interacting protein. In some embodiments, the ral interacting protein is at least one of RLIP76 or RALBP1 (RalA Binding Protein 1). In some embodiments, the composition overcomes deleterious effects of p53 gene loss.

In some embodiments, the compositions of the present disclosure bind to a site of a protein. In some embodiments, the site is a GTPase binding site of a protein. In some embodiments, the site is at ARG_232 in RLIP76. In some embodiments, the binding blocks the activity of RLIP76.

In some embodiments, the compositions of the present disclosure have a high docking score and high affinity for binding to the site of a protein. In some embodiments, the composition has an anti-proliferative effect on cancer cell lines. In some embodiments, chemical scaffolds of the compounds disclosed herein can be utilized to prepare derivatives. In some embodiments, the derivatives have improved docking scores and greater anticancer activity.

Method for Treating and/or Preventing Cancer

The compositions of the present disclosure may be utilized to treat and/or prevent cancer in various manners. For instance, in some embodiments, the compositions of the present disclosure treat and/or prevent cancer by binding to and inhibiting RLIP76.

Various methods may also be utilized to administer the compositions the present disclosure to a subject. For instance, in some embodiments, the administering can include, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, or combinations thereof.

In some embodiments, the administering is conducted at a low concentration of the compositions of the present disclosure. In some embodiments, the administering further includes co-administering to the subject one or more therapeutic compositions.

The methods of the present disclosure can be utilized to treat various types of cancer. For instance, in some embodiments, the cancer includes, without limitation, lung cancer, breast cancer, cancers associated with a an over-expression of a ral interacting protein, or combinations thereof.

In some embodiments, the cancer is breast cancer. In some embodiments, the breast cancer can include, without limitation, triple-negative breast cancer, ductal carcinoma in situ, invasive ductal carcinoma, tubular carcinoma of a breast, medullary carcinoma of a breast, mucinous carcinoma of a breast, papillary carcinoma of a breast, cribriform carcinoma of a breast, invasive lobular carcinoma, inflammatory breast cancer, lobular carcinoma in situ, male breast cancer, molecular subtypes of breast cancer, Paget's disease of a nipple, phyllodes tumors of a breast, metastatic breast cancer, or combinations thereof. In some embodiments, the breast cancer is triple-negative breast cancer.

In some embodiments, the cancer is lung cancer. In some embodiments, the lung cancer includes, without limitation, non-small cell lung cancer, squamous cell carcinoma, small cell lung cancer cell, or combinations thereof.

Without being bound by theory, the compositions and methods of the present disclosure can treat or prevent cancer through various mechanisms. For instance, in some embodiments, the compounds in the compositions of the present disclosure target cancer cells by inhibiting or binding to a ral interacting protein. In some embodiments, the ral interacting protein includes, without limitation, RLIP76, RALBP1 (RalA Binding Protein 1), or combinations thereof.

In some embodiments, the compounds of the present disclosure show high docking score for binding site centers of ral interaction proteins. For instance, in some embodiments, the compounds of the present disclosure show high docking score for the binding site center at ARG_232 in RALBP1.

In some embodiments, the compounds in the compositions of the present disclosure selectively target cancer cells. For instance, in some embodiments, the compounds in the compositions of the present disclosure selectively target cancer cells by reducing the proliferation of cancer cells relative to reducing the proliferation of normal cells. In some embodiments, the compounds in the compositions of the present disclosure have an anti-proliferative effect on cancer cells in vitro and in vivo.

The methods of the present disclosure can be utilized to treat cancer in various subjects. For instance, in some embodiments, the subject can include a mammal. In some embodiments, the subject is a human. In some embodiments, the subject is suffering from breast cancer, such as triple-negative breast cancer. In some embodiments, the subject is suffering from lung cancer.

In some embodiments, the methods and compositions of the present disclosure can be utilized to treat a cancer in a subject. In some embodiments, the methods and compositions of the present disclosure can be utilized to prevent a cancer in a subject. In some embodiments, the methods and compositions of the present disclosure can be utilized to treat and prevent a cancer in a subject.

Applications and Advantages

The present disclosure can have various advantages. For instance, in some embodiments, the compositions and methods of the present disclosure allow for a targeted therapy to treat and/or prevent triple-negative breast cancer cells by inhibiting the activity of RLIP76 and displaying minimal side effects.

In some embodiments, the compositions and methods presented herein can be utilized to kill breast cancer cells. In some embodiments, the compositions of the present disclosure can kill breast cancer cells at low concentrations, such as concentrations of less than 10 nmole, less than 5 nmole, less than 2 nmole, or less than 1 nmole.

As such, the compositions and methods of the present disclosure can be utilized in various manners and for various purposes. For instance, in some embodiments, the compositions and methods presented herein improve upon anticancer therapies, such as anticancer therapies that target the triple-negative breast cancer cell line. In some embodiments, the compositions and methods herein improve upon anticancer therapies, such as, but not limited to, therapies targeted towards cells expressing high levels of a ral interacting protein, for example, RLIP76.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Small Molecule Inhibitors of Ral Interacting Proteins (RLIP) for Inhibiting Breast Cancer Cells

This Example describes the identification of compounds that bind the ARG_232 region of Ral Interacting Proteins (RLIP76). RLIP76 is a stress-responsive protein subject to overexpression in some forms of breast cancer. When blocked, RLIP76 can overcome the deleterious effects of p53 gene loss more effectively.

Genetic defects in the p53 gene, a powerful tumor suppressor, can increase the risk of getting breast cancer and thereby cause the resulting cancers to become highly resistant to treatment. This Example identifies various suitable compounds to attach to the binding site center at ARG_232 in RLIP76 by a docking study. Compounds were identified to kill breast cancer cells at low concentrations by inhibiting RLIP, including, but not limited to, TTUHSC-SS-A4, TTUHSC-SS-B9, TTUHSC-SS-C2, TTUHSC-SS-C4, and TTUHSC-SS-E4, as will be discussed herein in further detail.

As also described herein, this Example shows that the aforementioned compounds have an anti-proliferative effect on breast cancer cell lines in vitro and in vivo in mouse xenografts. Similarly, this Example shows that these compounds have high docking scores, and therefore high affinity, for the binding site center at ARG_232 in RLIP76.

### Example 1.1. Inhibition of Breast Cancer Cells by TTUHSC-SS-A4

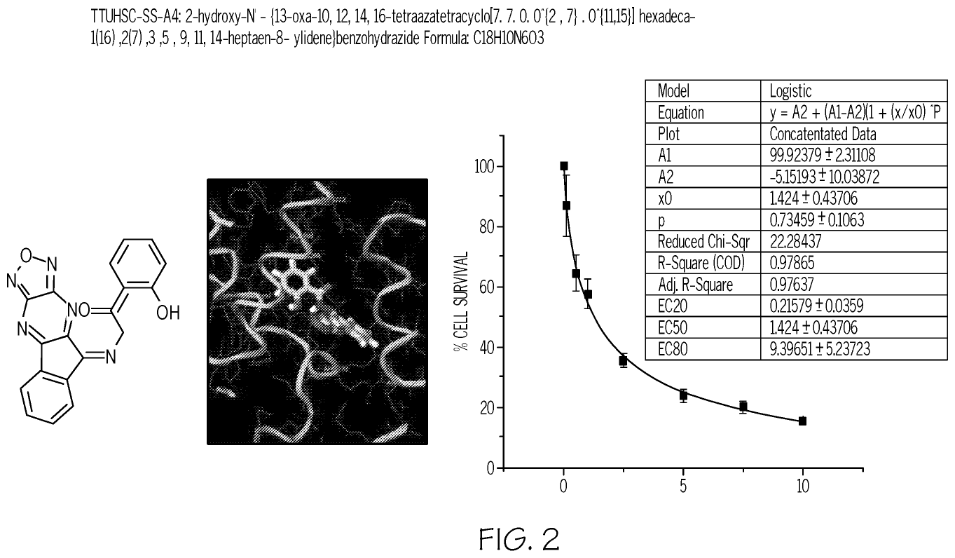

FIG. 2 illustrates a compound of the present disclosure termed TTUHSC-SS-A4 and corresponding data related to breast cancer cell inhibition by the compound.

### Example 1.2. Inhibition of Breast Cancer Cells by TTUHSC-SS-B9

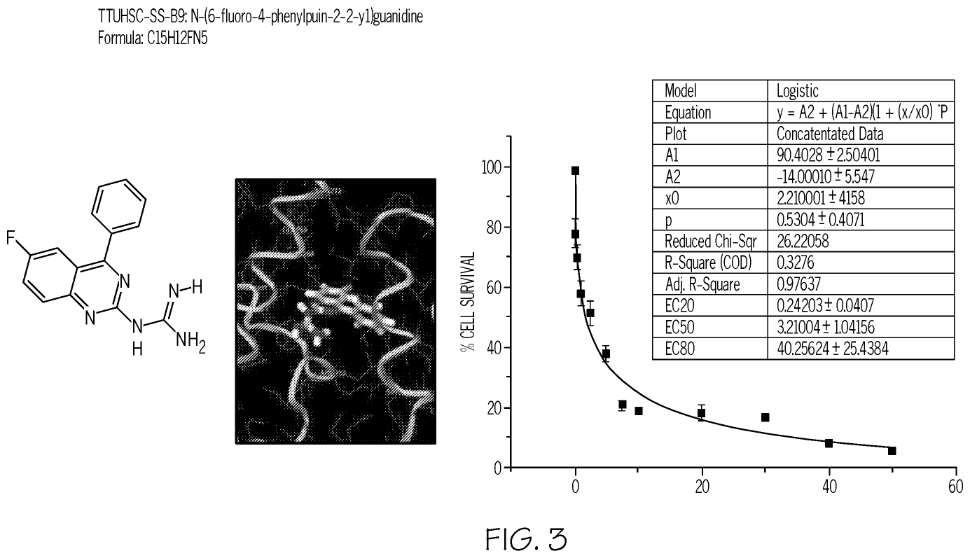

FIG. 3 illustrates a compound of the present disclosure termed TTUHSC-SS-B9 and corresponding data related to breast cancer cell inhibition by the compound.

### Example 1.3. Inhibition of Breast Cancer Cells by TTUHSC-SS-C2

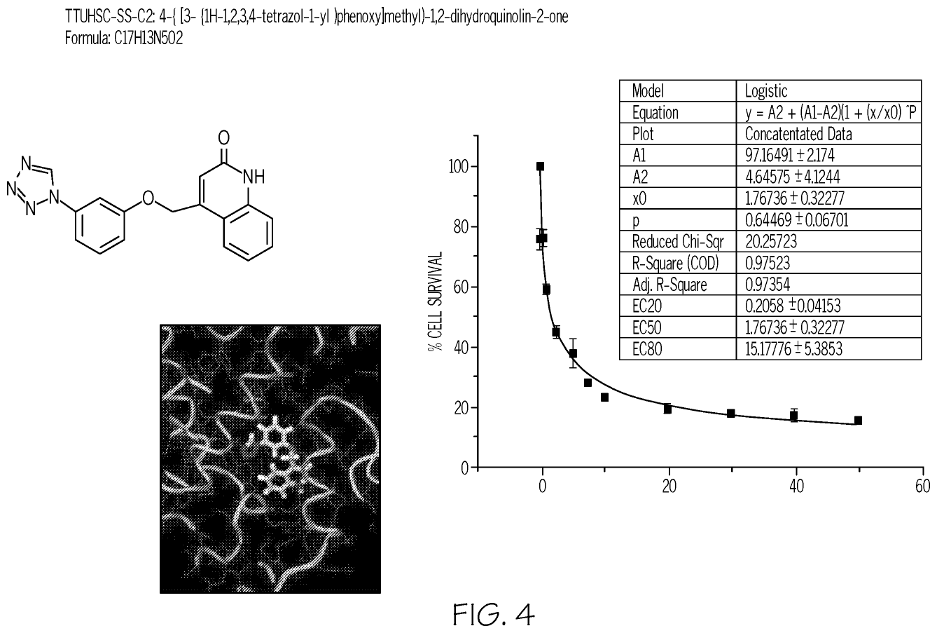

FIG. 4 illustrates a compound of the present disclosure termed TTUHSC-SS-C2 and corresponding data related to breast cancer cell inhibition by the compound.

### Example 1.4. Inhibition of Breast Cancer Cells by TTUHSC-SS-C4

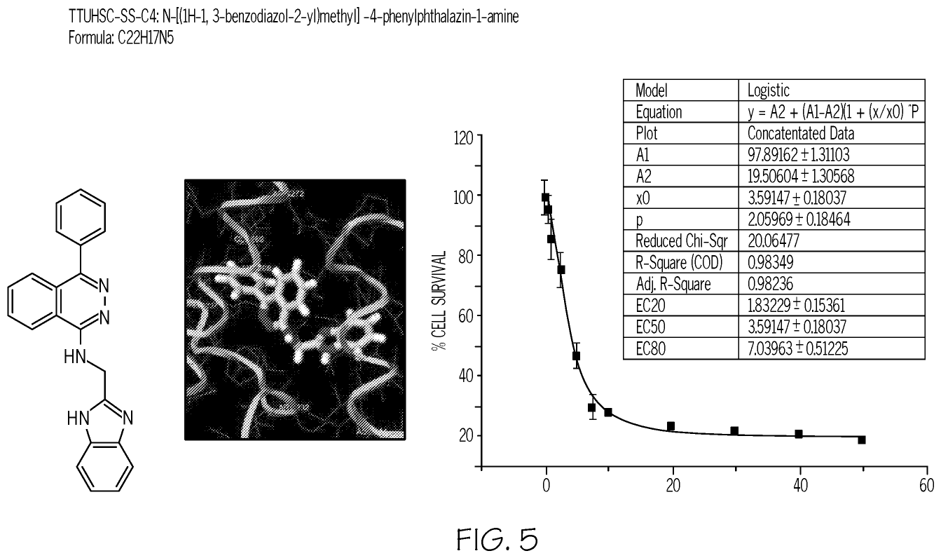

FIG. 5 illustrates a compound of the present disclosure termed TTUHSC-SS-C4 and corresponding data related to breast cancer cell inhibition by the compound.

### Example 1.5. Inhibition of Breast Cancer Cells by TTUHSC-SS-E4

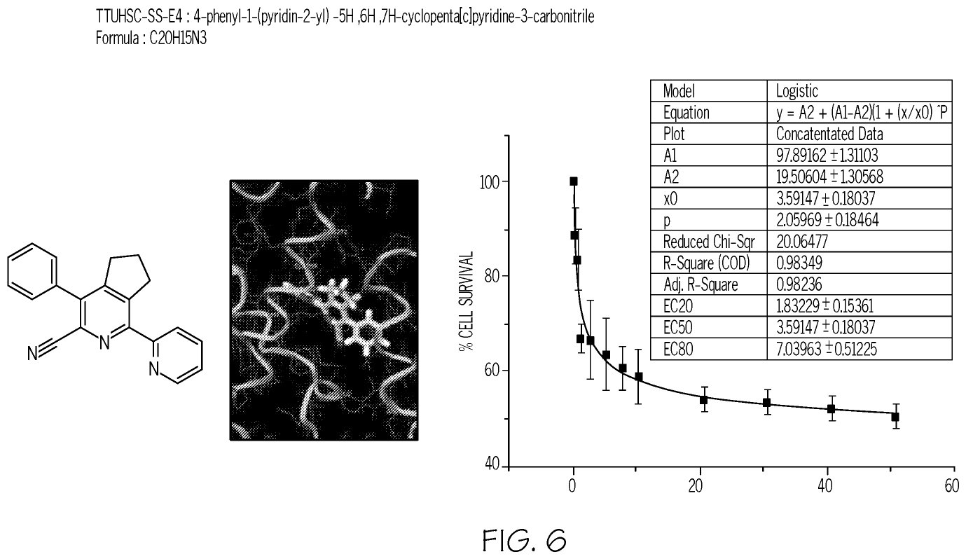

FIG. 6 illustrates a compound of the present disclosure termed TTUHSC-SS-E4 and corresponding data related to breast cancer cell inhibition by the compound.

### Example 1.6. Compound Testing

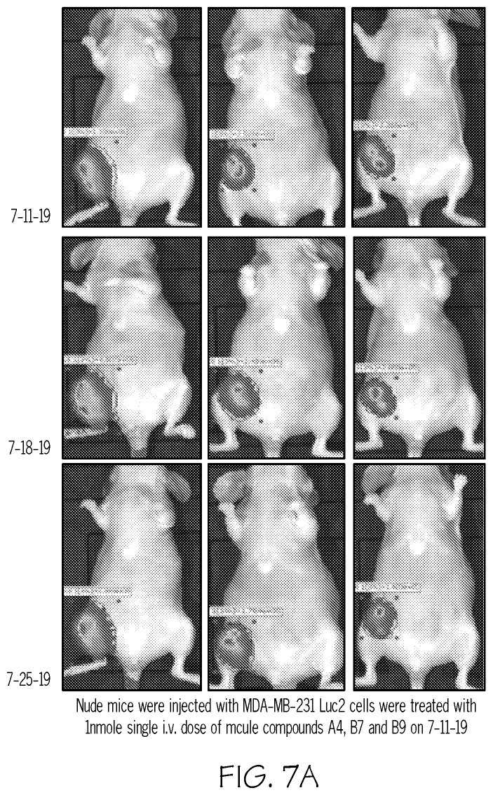

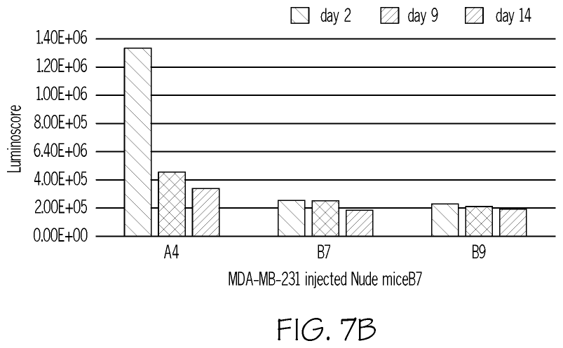

FIG. 7A illustrates images of nude mice after they were injected with MDA-MB-231 Luc2. The mice were then treated with 1 nmole single i.v. dose Mcule compounds A4, B7 and B9. FIG. 7B provides data related to Mcule compound testing.

### Example 2. Small Molecule Inhibitors of RLIP for Inhibiting Lung Cancer Cells

This Example relates to the use of various compounds that specifically target lung cancer cell lines, including non-small cell lung cancer cell lines, squamous cell carcinoma cell lines, and small cell lung cancer cell lines. Applicant identified the most suitable compounds for binding site center at ARG_232 in RLIP76 (2mbg.pdb) by docking studies. Applicant found that five of the compounds killed all four of the tested lung cancer cells at very low concentrations. However, lung bronchioepithelial cells (HLBEC) were less affected.

### Example 2.1. Inhibition of Lung Cancer Cells by TTUHSC-SS-A4

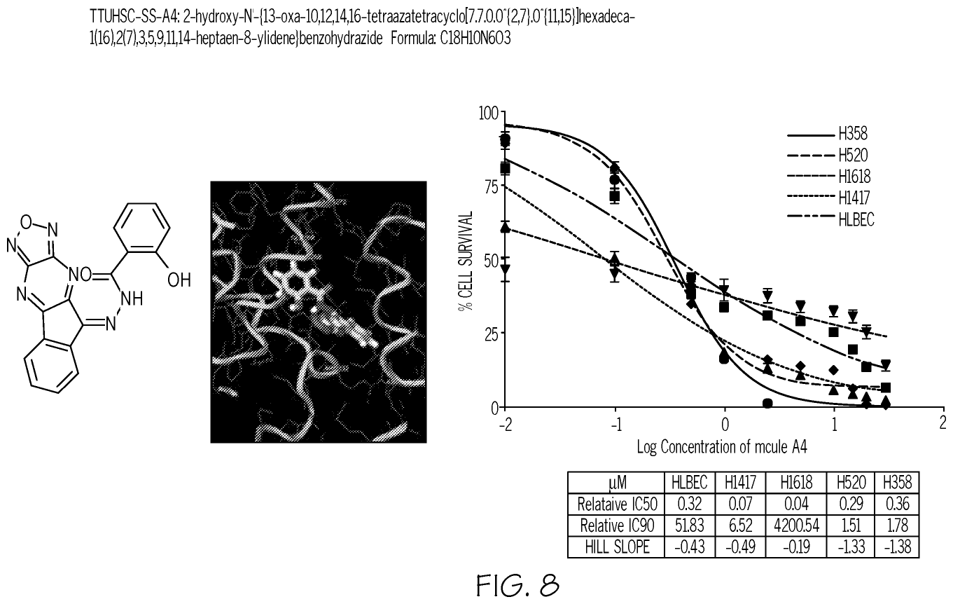

FIG. 8 illustrates a compound of the present disclosure termed TTUHSC-SS-A4 and corresponding data related to lung cancer cell inhibition by the compound.

### Example 2.2. Inhibition of Lung Cancer Cells by TTUHSC-SS-B9

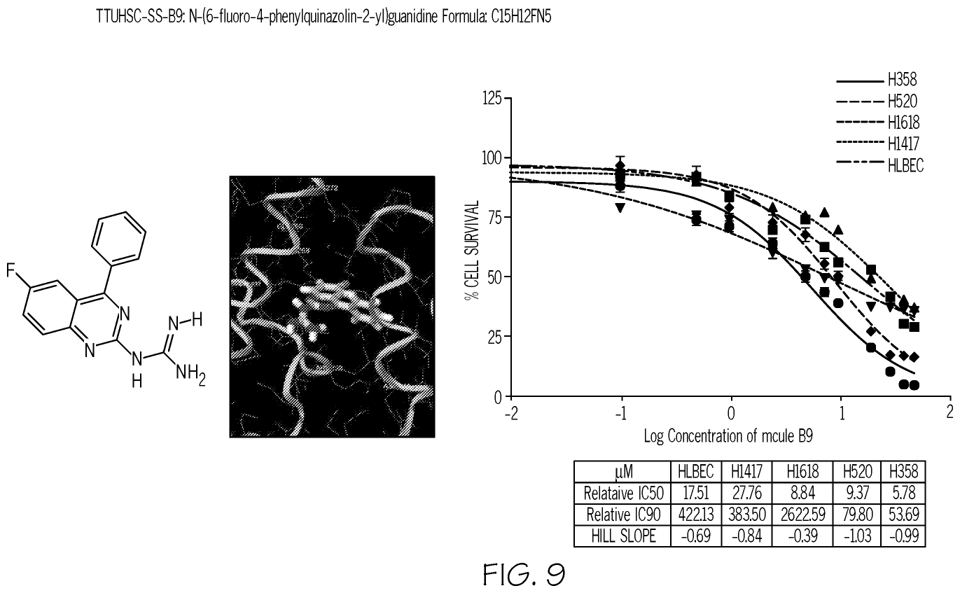

FIG. 9 illustrates a compound of the present disclosure termed TTUHSC-SS-B9 and corresponding data related to lung cancer cell inhibition by the compound.

### Example 2.3. Inhibition of Lung Cancer Cells by TTUHSC-SS-C2

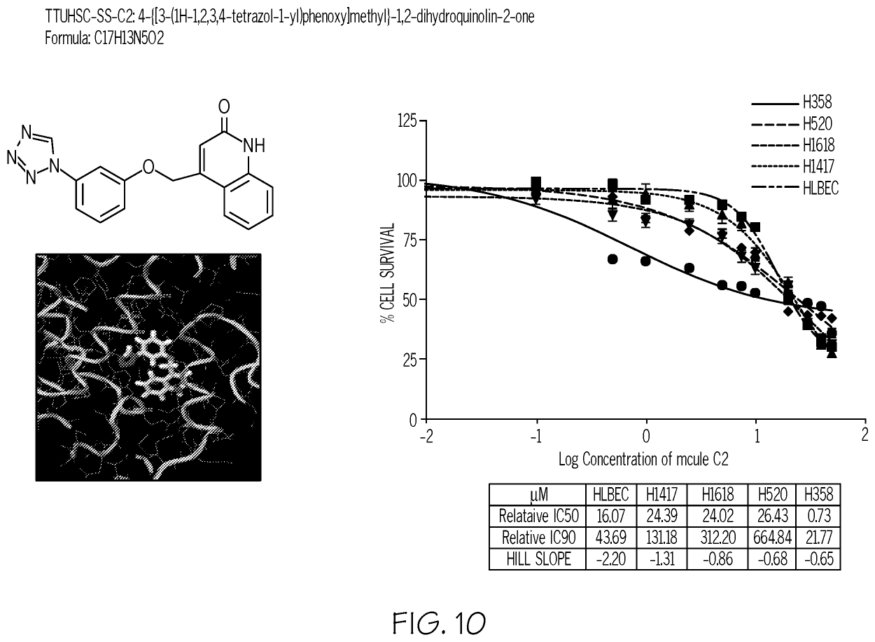

FIG. 10 illustrates a compound of the present disclosure termed TTUHSC-SS-C2 and corresponding data related to lung cancer cell inhibition by the compound.

### Example 2.4. Inhibition of Lung Cancer Cells by TTUHSC-SS-C4

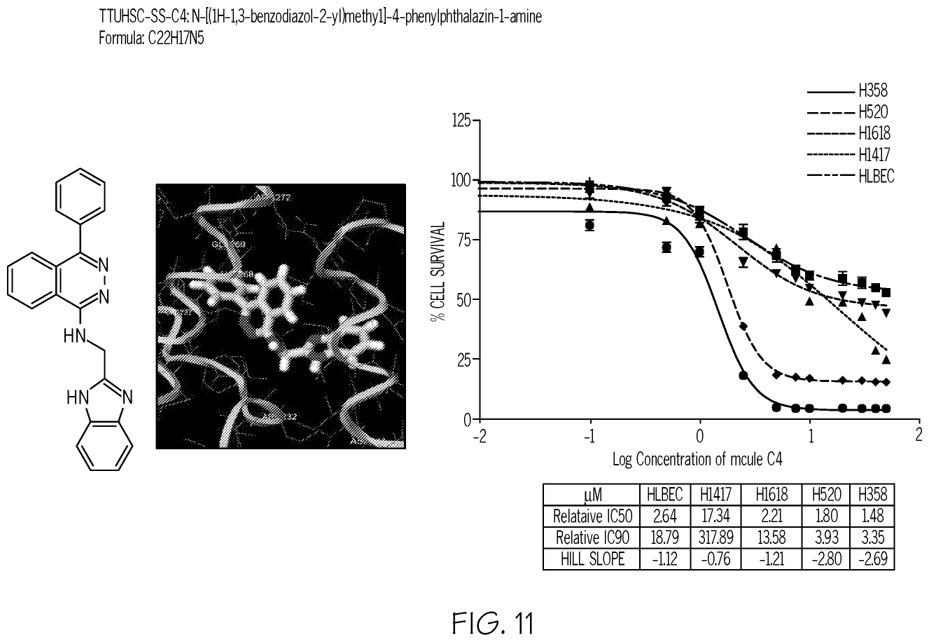

FIG. 11 illustrates a compound of the present disclosure termed TTUHSC-SS-C4 and corresponding data related to lung cancer cell inhibition by the compound.

### Example 2.5. Inhibition of Lung Cancer Cells by TTUHSC-SS-E4

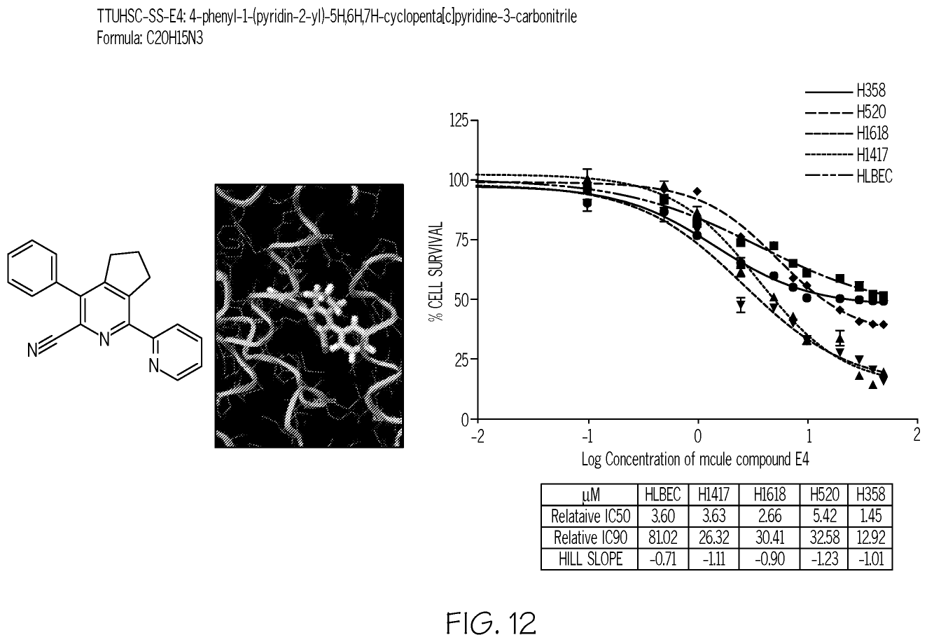

FIG. 12 illustrates a compound of the present disclosure termed TTUHSC-SS-E4 and corresponding data related to lung cancer cell inhibition by the compound.

In sum, this Example illustrated that various compounds have a strong anti-proliferative effect on lung cancer cell lines but minimal effects on normal lung epithelial cells. These compounds were also shown to have high docking score for binding site center at ARG_232 in RALBP1 (RalA Binding Protein 1), a gene over expressed in some aggressive forms of lung cancer.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treating or preventing cancer in a subject, the method comprising:
administering to the subject a composition,
wherein the composition comprises at least one compound selected from the group consisting of: 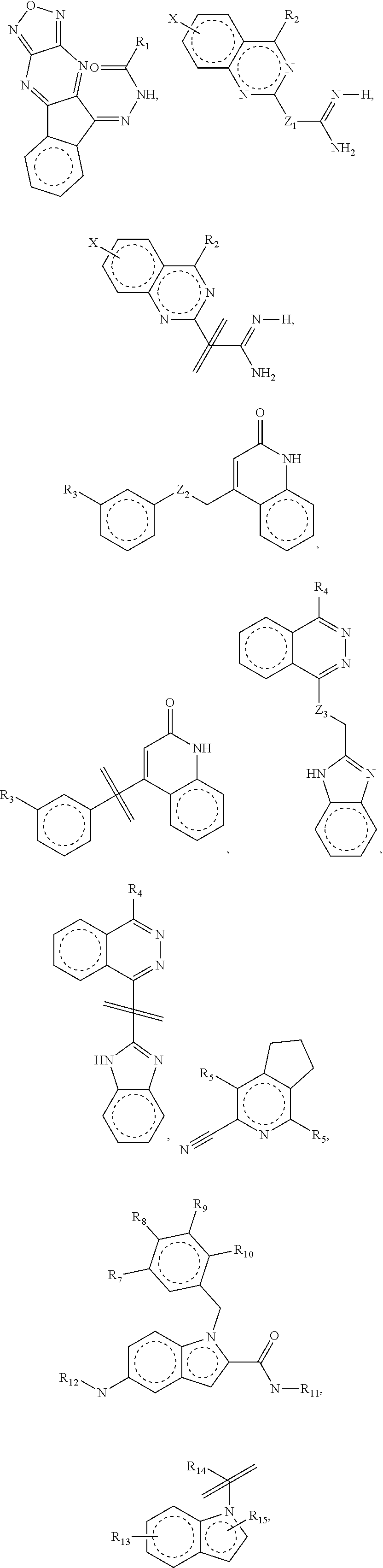
derivatives thereof, or combinations thereof;
wherein R₁, R₂, R₃, R₄, R₅, R₆, R₇, R₈, R₉, R₁₀, R₁₁, R₁₂, R₁₃, R₁₄, and R₁₅ are each independently selected from the group consisting of an alkane, an alkene, an alkyne, a benzene derivative, an alkyl, an alkenyl, an alkynyl, a phenol, a phenyl, a haloalkane, a fluoroalkane, a chloroalkane, a bromoalkane, an iodoalkane, an alcohol, a ketone, an aldehyde, an acyl halide, a carbonate, a carboxylic acid, an ester, a methoxy, a hydroperoxide, a peroxide, an ether, a hemiacetal, a hemiketal, an acetal, a ketal, an orthoester, a heterocycle, an orthocarbonate ester, an organic acid anhydride, a hydroxyl, a carbonyl, an aldehyde, a haloformyl, a carbonate ester, a anhydride, an amide, an amine, an imine, an imide, an azide, an azo, a cyanate, a nitrate, a nitrile, a nitrite, a nitro, a nitroso, an oxime, a pyridine, a pyridine derivative, a carbamate ester, a carbamate, a thiol, a sulfide, a disulfide, a sulfoxide, a sulfone, a sulfinic acid, a sulfonic acid, a sulfonate ester, a thiocyanate, a thioketone, a thial, a thiocarboxylic acid, a thioester, a dithiocarboxylic acid, a dithiocarboxylic acid ester, a phosphine, a phosphonic acid, a phosphate, a phosphodiester, H, C, F, CH₃, AcHN, CONH₂, CF₃, MeHN, 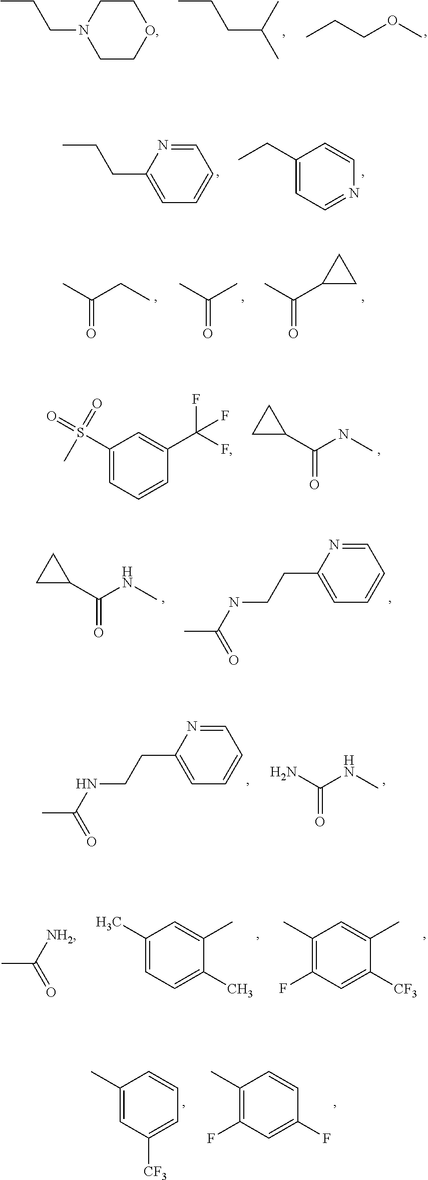
derivatives thereof, or combinations thereof;
wherein Z₁, Z₂, and Z₃ are each independently selected from the group consisting of O, NH, CH₂, S, Se, or combinations thereof; and
wherein X selected from the group consisting of H, a halogen, fluorine, chlorine, bromine, iodine, or combinations thereof.

2. The method of claim 1, wherein the compound is selected from the group consisting of: 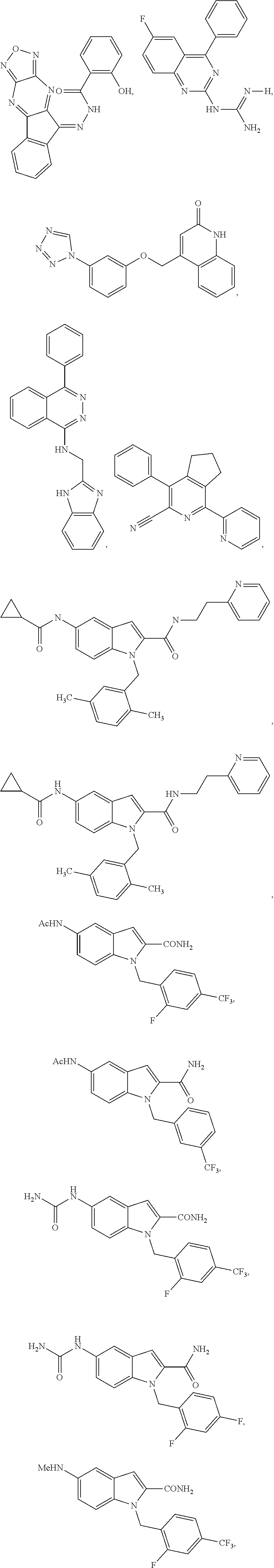
derivatives thereof, or combinations thereof.

3. The method of claim 1, wherein the compound is selected from the group consisting of: 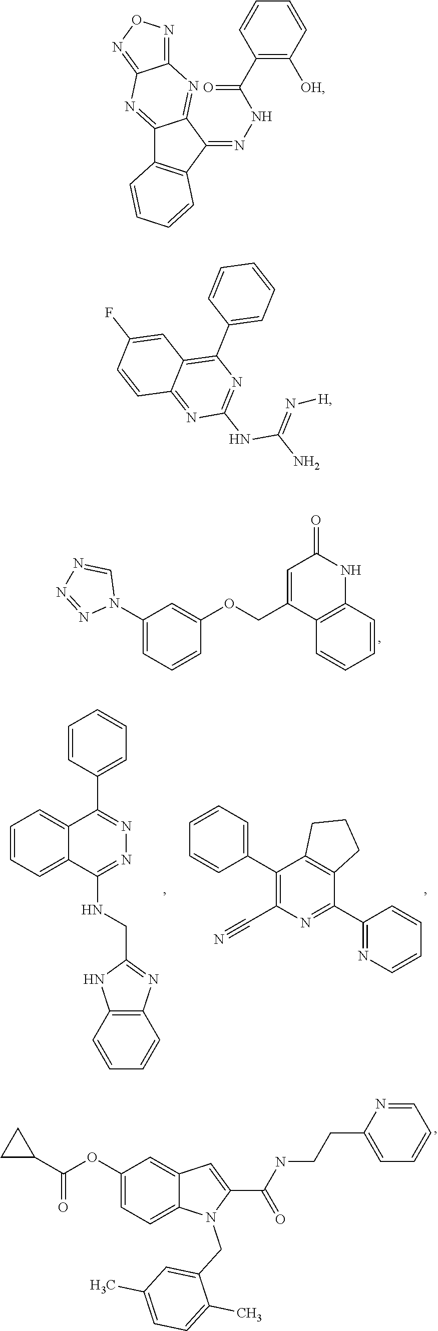
or combinations thereof.

4-8. (canceled)

9. The method of claim 1, wherein the administering comprises intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, or combinations thereof.

10. The method of claim 1, wherein the administering comprises co-administering to the subject one or more therapeutic compositions.

11. The method of claim 1, wherein the cancer is selected from the group consisting of lung cancer, breast cancer, cancers associated with an over-expression of a ral interacting protein, or combinations thereof.

12. The method of claim 1, wherein the cancer is breast cancer, wherein the breast cancer is selected from the group consisting of triple-negative breast cancer, ductal carcinoma in situ, invasive ductal carcinoma, tubular carcinoma of a breast, medullary carcinoma of a breast, mucinous carcinoma of a breast, papillary carcinoma of a breast, cribriform carcinoma of a breast, invasive lobular carcinoma, inflammatory breast cancer, lobular carcinoma in situ, male breast cancer, molecular subtypes of breast cancer, Paget's disease of a nipple, phyllodes tumors of a breast, metastatic breast cancer, or combinations thereof.

13. (canceled)

14. The method of claim 12, wherein the breast cancer is triple-negative breast cancer.

15. The method of claim 1, wherein the cancer is lung cancer.

16. The method of claim 15, wherein the lung cancer is selected from the group consisting of non-small cell lung cancer, squamous cell carcinoma, small cell lung cancer cell, or combinations thereof.

17. The method of claim 1, wherein the compound targets cancer cells by inhibiting or binding to a ral interacting protein.

18. The method of claim 17, where the ral interacting protein is selected from the group consisting of RLIP76, RALBP1 (RalA Binding Protein 1), or combinations thereof.

19. The method of claim 1, wherein the compound selectively targets cancer cells by reducing the proliferation of cancer cells relative to reducing the proliferation of normal cells.

20. The method of claim 1, wherein the subject is selected from the group consisting of a mammal, a human, or combinations thereof.

21. The method of claim 1, wherein the subject is a human.

22. The method of claim 1, wherein the method is utilized to treat the cancer in the subject.

23. The method of claim 1, wherein the method is utilized to prevent the cancer in the subject.

24. A composition, wherein the composition comprises at least one compound selected from the group consisting of: 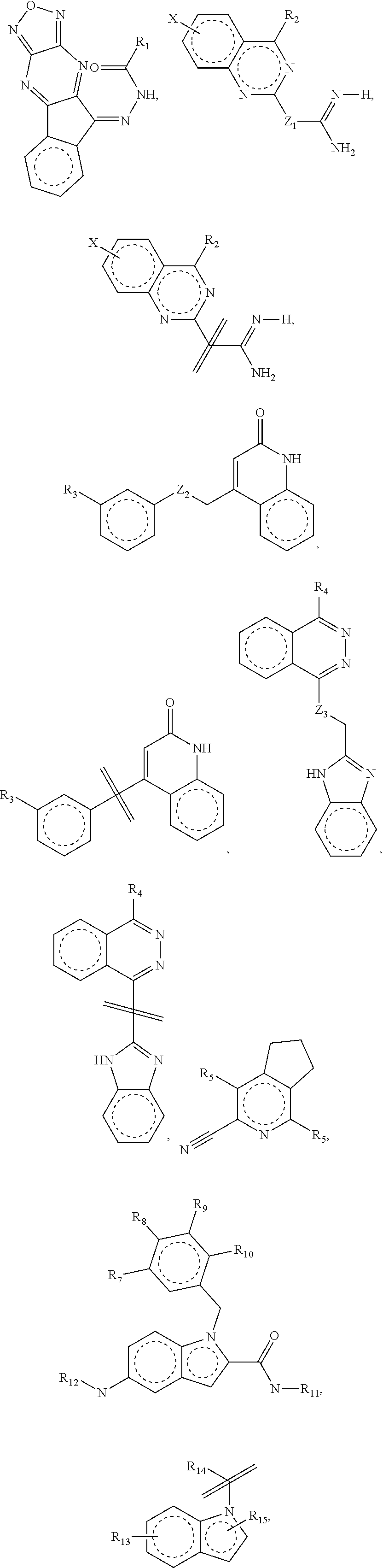
derivatives thereof, or combinations thereof;
wherein R₁, R₂, R₃, R₄, R₅, R₆, R₇, R₈, R₉, R₁₀, R₁₁, R₁₂, R₁₃, R₁₄, and R₁₅ are each independently selected from the group consisting of an alkane, an alkene, an alkyne, a benzene derivative, an alkyl, an alkenyl, an alkynyl, a phenol, a phenyl, a haloalkane, a fluoroalkane, a chloroalkane, a bromoalkane, an iodoalkane, an alcohol, a ketone, an aldehyde, an acyl halide, a carbonate, a carboxylic acid, an ester, a methoxy, a hydroperoxide, a peroxide, an ether, a hemiacetal, a hemiketal, an acetal, a ketal, an orthoester, a heterocycle, an orthocarbonate ester, an organic acid anhydride, a hydroxyl, a carbonyl, an aldehyde, a haloformyl, a carbonate ester, a anhydride, an amide, an amine, an imine, an imide, an azide, an azo, a cyanate, a nitrate, a nitrile, a nitrite, a nitro, a nitroso, an oxime, a pyridine, a pyridine derivative, a carbamate ester, a carbamate, a thiol, a sulfide, a disulfide, a sulfoxide, a sulfone, a sulfinic acid, a sulfonic acid, a sulfonate ester, a thiocyanate, a thioketone, a thial, a thiocarboxylic acid, a thioester, a dithiocarboxylic acid, a dithiocarboxylic acid ester, a phosphine, a phosphonic acid, a phosphate, a phosphodiester, H, C, F, CH₃, AcHN, CONH₂, CF₃, MeHN, 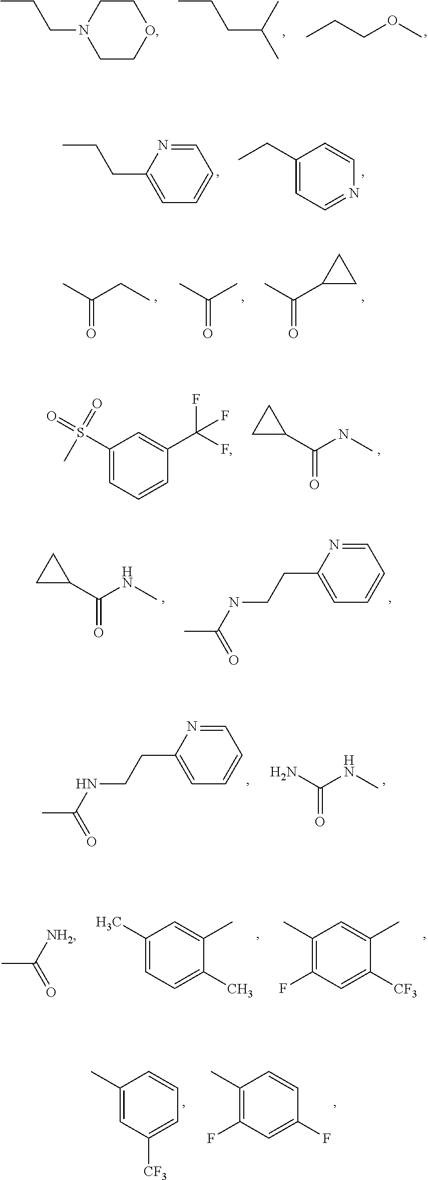
derivatives thereof, or combinations thereof;
wherein Z₁, Z₂, and Z₃ are each independently selected from the group consisting of O, NH, CH₂, S, Se, or combinations thereof; and
wherein X selected from the group consisting of H, a halogen, fluorine, chlorine, bromine, iodine, or combinations thereof.

25. The composition of claim 24, wherein the compound is selected from the group consisting of: 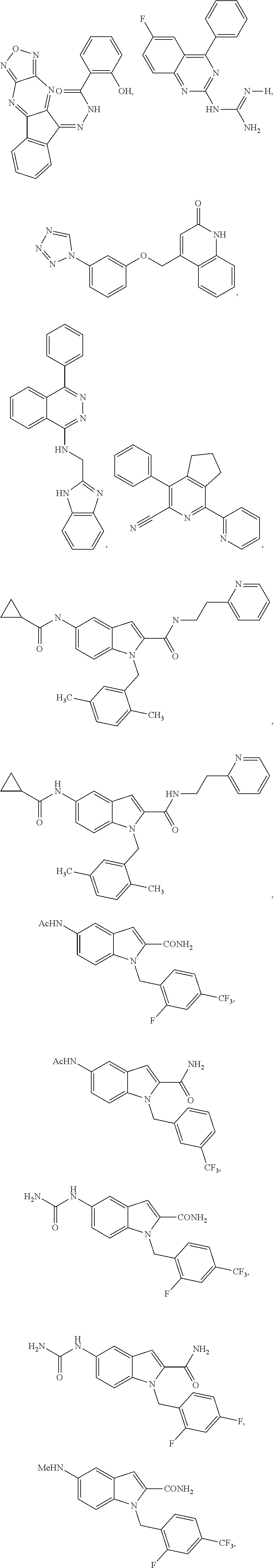
derivatives thereof, or combinations thereof.

26. The composition of claim 24, wherein the compound is selected from the group consisting of: 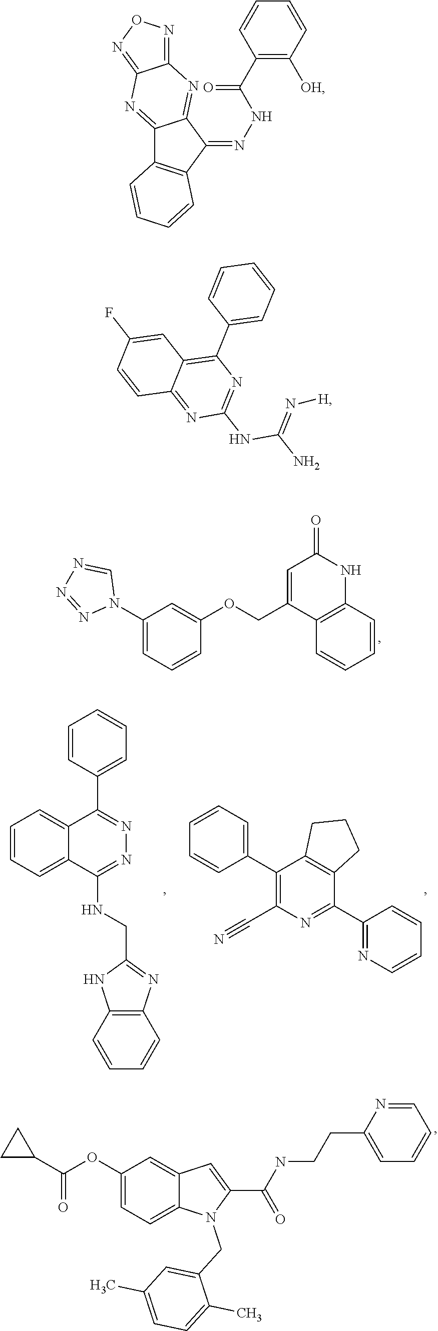
or combinations thereof.

27-32. (canceled)
